(19) United States
(12) Patent Application Publication
(10) Pub. No.: US 2006/0206126 A1
(43) Pub. Date: Sep. 14, 2006

(54) Blade for use in corneal surgery and corneal surgical apparatus having the same

(76) Inventors: Masahiro Sugimura, Aichi (JP); Kengo Asai, Aichi (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/344,145

(22) Filed: Feb. 1, 2006

(30) Foreign Application Priority Data
Feb. 1, 2005 (JP) P2005-025606

(51) Int. Cl. A61F 9/00

(52) U.S. Cl. 606/166

(57) ABSTRACT

A blade for used in corneal surgery for incising and separating a corneal epithelium into a flap shape, the blade includes: an edge tip portion including a distal end and two upper and lower edge tip surfaces. An angle formed between the two edge tip surfaces is not smaller than 60° and smaller than 140°, and a height of the edge tip portion is not smaller than 3 μm.

### BACKGROUND

This invention relates to a blade for used in corneal surgery, which is suitably used for incising and separating a corneal epithelium into a flap shape, and a corneal surgical apparatus having the same.

As a method of corneal refractive surgery, LASIK (Laser Assisted In-Situ Keratomileusis) is known in which a portion of about 150 μm in thickness of a cornea which extends from a corneal epithelium to a corneal stroma is incised in layer with one end (hinge) of the cornea left uncut to form a flap, and the formed flap is restored to its original state after subjecting a quantity to be corrected of the corneal stroma to ablation with a laser beam. In this LASIK, a corneal surgical apparatus called “keratome” for incising a cornea in layer is used.

In recent years, a method called LASEK (Laser Epithelial Keratomileusis) has also been proposed in which an annular incision is made into a corneal epithelium with a hinge left uncut by using an epi-trephine, the corneal epithelium is immersed in alcohol and the like to expand the same, the corneal epithelium is then separated from a Bowman's membrane with a golf knife to form a flap, a corneal stroma is subjected to ablation with a laser beam and the flap is restored to its own condition. This LASEK can also be applied to a thin cornea to which LASIK cannot be applied. However, the use of alcohol causes the cornea to damage in some cases, and formation of the flap with the golf knife and the like takes much labor and time.

### SUMMARY

In view of the above-described problems encountered in the related techniques, it is a technical object of the present invention to provide a blade for used in corneal surgery capable of forming a corneal epithelium flap easily and smoothly without using alcohol and the like, and capable of forming an edge thereof easily, and a corneal surgical apparatus having the same.

To solve these problems, the present invention is characterized in that the invention has the following structure.

(1) A blade for used in corneal surgery for incising and separating a corneal epithelium into a flap shape, the blade comprising:

an edge tip portion including a distal end and two upper and lower edge tip surfaces,

wherein an angle formed between the two edge tip surfaces is not smaller than 60° and smaller than 140°, and

wherein a height of the edge tip portion is not smaller than 3 μm.

(2) The blade according to (1), wherein the angle formed between the two edge tip surfaces is not smaller than 70° and not larger than 120°.

(3) The blade according to (1), wherein an angle of the lower edge tip surface with respect to a center line passing the distal end is larger than an angle of the center line with respect to a horizontal plane in the blade at the time of incision and separation of the corneal epithelium.

(4) A corneal surgical apparatus comprising:

holding means for oscillatingly holding the blade according to (1); and

oscillating means for oscillating the blade, wherein the corneal epithelium is incised and separated with the oscillating blade.

(5) A corneal surgical apparatus comprising: an incising unit provided with a blade holder for holding the blade according to (1) oscillatingly in a predetermined direction; and a vibrating mechanism that has a driving power source, converts power from the power source into kinetic energy in a predetermined direction, transmits the kinetic energy to the blade, and oscillates the blade, wherein the corneal epithelium is incised and separated with the oscillating blade.

(6) A blade for used in a corneal surgery, for incising and separating a corneal epithelium into a flap shape, the blade comprising;

an edge tip portion including a distal end flat surface, and two upper and lower edge tip surfaces.

wherein an angle formed between the two edge tip surfaces is not smaller 30° and smaller than 80°, and

wherein a height of the distal end flat surface is not smaller than 1 μm and not larger than 3 μm.

(7) The blade according to (6), wherein the angle formed between the two edge tip surfaces is not smaller than 50° and not larger than 70°.

(8) A corneal surgical apparatus comprising: holding means for oscillatingly holding the blade according to (6); and oscillating means for oscillating the blade, wherein the corneal epithelium is incised and separated with the oscillating blade.

(9) A corneal surgical apparatus comprising: an incising unit provided with a blade holder for holding the blade according to (6) oscillatingly in a predetermined direction; and a vibrating mechanism that has a driving power source, converts power from the power source into kinetic energy in a predetermined direction, transmits the kinetic energy to the blade, and oscillates the blade, wherein the corneal epithelium is incised and separated with the oscillating blade.

### BRIEF DESCRIPTION OF THE DRAWINGS

FIG. 1 is a schematic construction diagram of a corneal surgical apparatus according to an embodiment of the present invention.

FIG. 2 is an enlarged view of FIG. 1 showing an incising unit and a suction unit.

FIG. 3 is a diagram for describing the shape of a blade.

FIG. 4 is an enlarged view for describing a first shape of an edge tip portion of the blade.

FIG. 5 is a diagram showing the results of experiments on the edge tip portion having the first shape.

FIG. 6 is an enlarged view for describing a second shape of the edge tip portion of the blade.

FIG. 7 is a diagram showing results of experiments on the edge tip portion having the second shape.

FIG. 8 is a diagram showing results of experiments on the edge tip portion having the first shape.

### DETAILED DESCRIPTION OF THE PREFERRED EMBODIMENTS

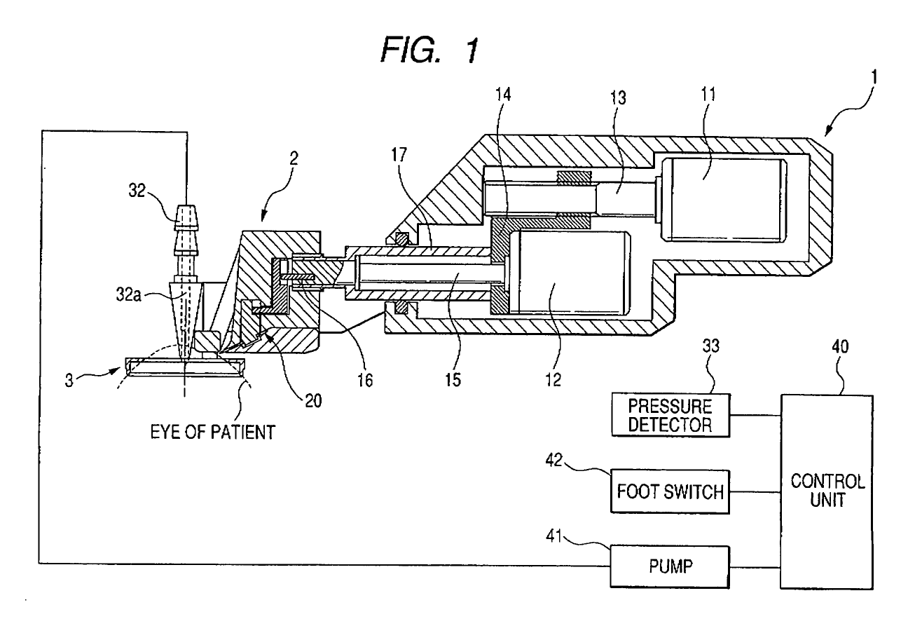

An embodiment of the present invention will now be described on the basis of the drawings. FIG. 1 is a schematic construction diagram of a corneal surgical apparatus according to an embodiment of the present invention.

A body 1 of the corneal surgical apparatus is provided at a front side (left side of FIG. 1) thereof with a suction unit 3 for fixing the apparatus to an eye of a patient, and an incising unit 2 having a blade 20 for incising and separating a corneal epithelium and moving linearly on the suction unit 3.

A translation motor 11 for linearly moving the incising unit 2 (blade 20) in an incising and separating direction is fixedly mounted on the body 1, and an oscillation motor 12 for oscillating the blade 20 at a high speed in a direction of the width of the blade 20 is movably mounted on the body 1. A feed screw 13 having a threaded portion corresponding to a distance at which the incising unit 2 is moved is fixed to a rotary shaft of the motor 11. The motor 12 and a rear end of a connecting member 17 to which the incising unit 2 is connected are fixed to a connecting member 14 meshed with the feed screw 13. With this structure, the motor 12 and the connecting member 17 are moved forward and backward in an axial direction of the feed screw 13 via the feed screw 13 and the connecting member 14 due to forward and rearward rotations of the motor 11, so that the incising unit 2 connected to a front end of the connecting member 17 is moved forward and backward in the same direction. A rear end of a rotary shaft 15 is fixed to a rotary shaft of the motor 12, and the rotary shaft 15 is held rotatably on the connecting member 17. An eccentric pin 16 is provided in a position offset from the center of rotation of a front end of the rotary shaft 15.

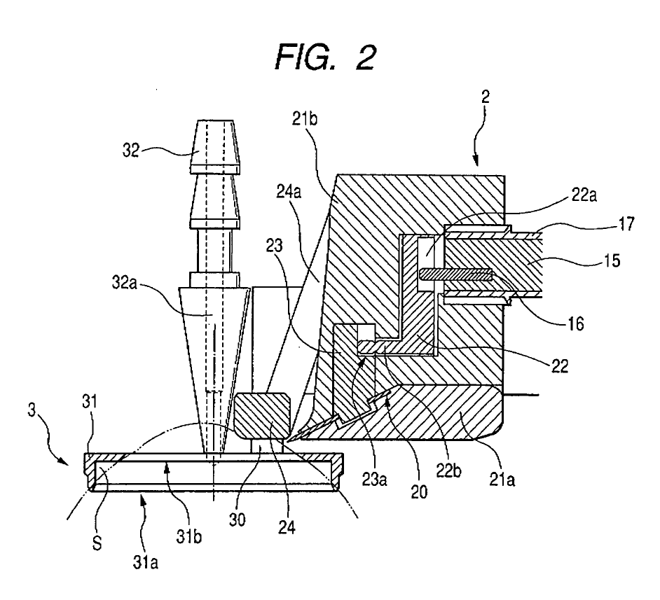

FIG. 2 is an enlarged view of FIG. 1 showing the incising unit 2 and a suction unit 3. The incising unit 2 includes a blade holder 21a and a holder block 21b which hold the blade 20 so as to allow oscillation, a first oscillation transmission member 22 for transmitting oscillation generated by the eccentric pin 16 to the blade 20, a second oscillation transmission member 23 for transmitting oscillation of the first transmission member 22 to the blade 20, and a cornea applanating member 24 fixed to the holder block 21b by a fixing member 24a. The holder block 21b is provided with a hole into which the rotary shaft 15 is inserted, and in which the front end of the connecting member 17 is fixed.

As the blade 20, a metal blade using stainless steel and the like for an edge, a mineral blade using diamond, sapphire and the like for an edge, a resin blade, such as plastic and the like are employed. The blade 20 is oscillatingly held between the blade holder 21a and the holder block 21b at an angle (incising angle) of not smaller than about 15° and not larger than about 30° with respect to a horizontal plane. The angle of the blade 20 in this apparatus is set about 25°. The shape of the edge of the blade 20 will be described later.

The first transmission member 22 is held movable in a receiving recess formed in the holder block 21b in a lateral (left and right) direction perpendicular to the direction of movement of the incising unit 2. The first transmission member 22 is held by the holder block 21b in upper and lower sides thereof. The first transmission member 22 is provided with a vertical recess 22a engaged with the eccentric pin 16. When the rotary shaft 15 is rotated by rotation of the motor 12, lateral kinetic energy is exerted on the first transmission member 22 due to circumferential movement of the eccentric pin 16 engaged with the vertical recess 22a. This causes the first transmission member 22 to be laterally oscillated.

The second transmission member 23 is held laterally movable in a receiving recess formed in the holder block 21b. The second transmission member 23 is held by the holder block 21b in the upper side thereof, and by the blade holder 21a in the lower side thereof. The first transmission member 22 is provided at a lower portion thereof with a projection 22b projecting toward the blade 20, and the second transmission member 23 is provided with a vertical recess 23a engaged with the projection 22b. When the first transmission member 22 is laterally oscillated by a rotation of the rotary shaft 15 (circumferential oscillation of the eccentric pin 16), lateral kinetic energy is further exerted on the second transmission member 23 due to lateral oscillation of the projection 22b engaged with the vertical recess 23a. This causes the second transmission member 23 to be laterally oscillated, and the blade 20 fixed to the second transmission member 23 to be laterally oscillated.

The applanating member 24 is provided at the front side (left side in FIGS. 1 and 2) of the blade 20, and presses the cornea flat in accordance with linear movement of the incising unit 2 before incision and separation of the corneal epithelium by the blade 20. By incising and separating the corneal epithelium pressed flat by the applanating member 24 by the blade 20, a flap of the corneal epithelium separated from a Bowman's membrane is formed.

In order to separate the corneal epithelium from the Bowman's membrane, the edge of the blade 20 is positioned lower by a distance corresponding to the thickness of the corneal epithelium with respect to a lower surface of the applanating member 24. A distance between the lower surface of the applanating member 24 and the edge of the blade 20 is not smaller than about 100 μm and not larger than about 300 μm, and, in the embodiment, about 150 μm.

The suction unit 3 includes a fixing member 30, a suction ring 31, a suction pipe 32 and the like, and the suction ring 31 is fixed to the body 1 with the fixing member 30. The suction ring 31 has a substantially cylindrical shape having a U-shaped cross section, and includes a circular recess 31a for contacting the patient's eye, and an opening 31b concentric with the recess 31a. when the suction ring 31 is set on the patient's eye at the surgery, the cornea projects upward from the opening 31b, and a lower end portion of the suction ring 31 and an opened end portion of the opening 31b are engaged with the patient's eye, and a suction space S is secured owing to this engagement.

The suction pipe 32 is provided to the suction ring 31, and connected to a vacuum tube (not shown) extending to a pump 41. A suction passage 32a provided in an inner portion of the suction pipe 32 communicates with the recess 31a, and the air in the space S is sucked and discharged by the pump 41 via the suction passage 32a, so that the suction ring 31 is fixed to the patient's eye by suction.

The pressure detecting pipe (not shown) is provided in the suction ring 31, and the pipe is connected to a pressure detector 33 by a tube (not shown). The pressure detector 33 detects via the pressure detecting pipe air pressure in the space S in which the air is sucked by the pump 41. A control unit 40 controls operations of the motor 11, the motor 12, the pump 41 and the like on the basis of the air pressure detected by the pressure detector 33.

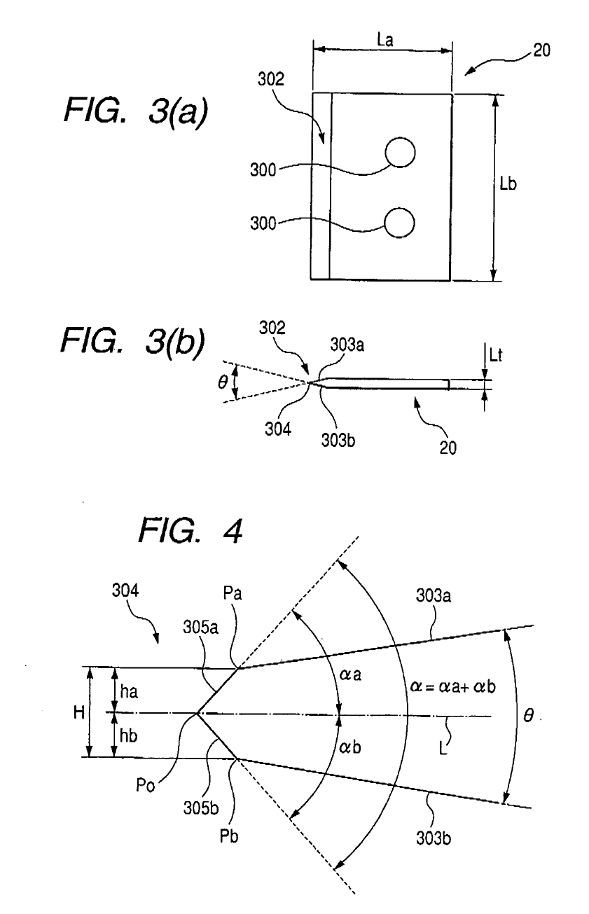

The shape of the blade 20 will now be described. FIG. 3(a) is a plan view of the blade 20, and FIG. 3(b) a sectional view of the blade 20. The blade 20 is about 8 mm in length La, about 13 mm in width Lb, and about 0.25 mm in thickness Lt of a base portion. Reference numerals 300 denote holes in which the second transmission members 23 are fitted. At a front portion of the blade 20, a blade edge 302 having upper and lower edge surfaces 303a and 303b, and an edge tip portion 304 having a distal end Po (refer to FIG. 4). The edge surfaces 303a and 303b are formed incliningly so as to form substantially equal angles with respect to a center line L passing the distal end Po. The edge surfaces 303a and 303b form an angle θ therebetween of not smaller than about 15° and not larger than about 40°, and, in the embodiment, about 19°. The angle θ formed between the edge surfaces 303a and 303b may be set in a stepped manner in 2 to 3 steps toward the edge tip portion 304.

FIG. 4 is an enlarged view for describing a first shape of the edge tip portion 304 of the blade 20. The edge tip portion 304 has two upper and lower edge tip surfaces 305a and 305b forming an angle α larger than the angle θ formed between the edge surfaces 303a and 303b. The reasons reside in that, when the angle of the edge tip portion of the blade is set large (obtuse), it becomes possible to form a flap incised and separated from the corneal epithelium only without incising the corneal stroma. In order to form the edge tip portion of the blade obtusely, the edge tip portion may be curved but it is difficult to form a curved portion.

The angle α of the edge tip portion 304 is set to a level at which the blade 20 incises the corneal epithelium but not the Bowman's membrane harder than the corneal epithelium. When the angle α is too small (too sharp), the blade 20 incises even the Bowman's membrane and also the corneal stroma thereunder. Conversely, when the angle α is too large (too obtuse), the corneal epithelium cannot be incised. This angle α is not smaller than about 60° and smaller than about 140°, and preferably not smaller than about 70° and not larger than about 120°. A height H of the edge tip portion 304 (measured from a boundary position Pa between the edge surface 305a and edge tip surface 303a to a boundary position Pb between the edge tip surface 305b and the edge surface 303b) is at least not smaller than about 3 μm, and preferably not smaller than about 5 μm.

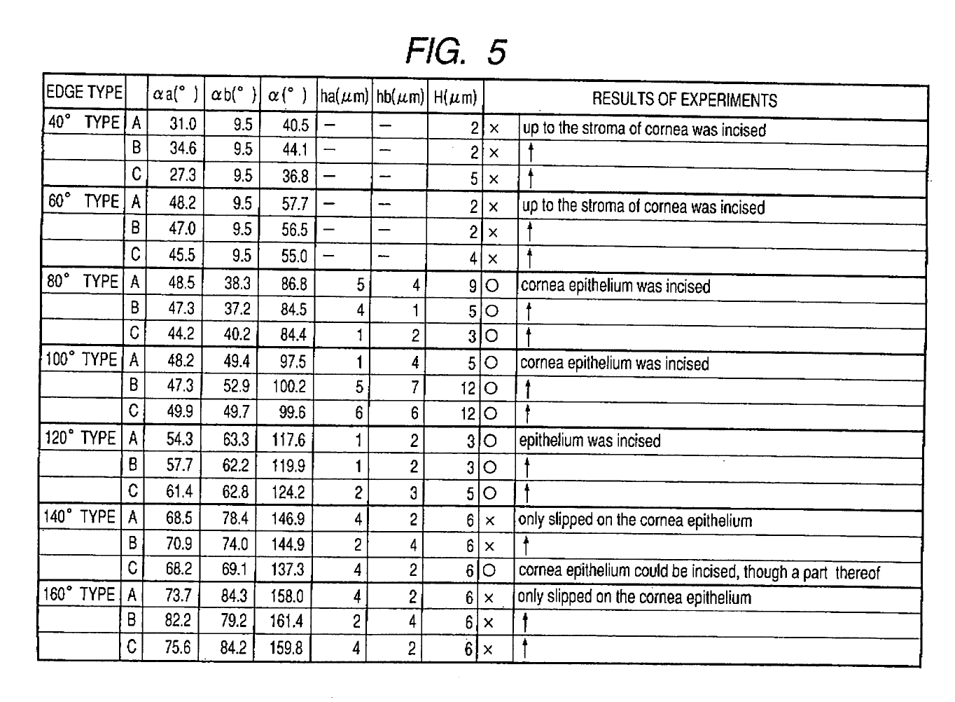

FIG. 5 is a diagram showing results of experiments for making sure an allowable range of the angle α of the edge tip portion 304 having the first shape. The experiments were conducted by using pig's eyes, dividing angles α into seven groups of about 40°, about 60°, about 80°, about 100°, about 120°, about 140° and about 160° and preparing blades 20 each of which has three kinds (A, B, C) of edge tip portions 304. The transaction speed of the blade 20 in the experiments is set to about 2 mm/sec and the number of oscillation was to about 9000 rpm.

Referring to FIG. 4 and FIG. 5, αa is an angle of the edge tip surface 305a with respect to the center line L, and αb is an angle of the edge tip surface 305b with respect to the center line L. Therefore, the angle α of the edge tip portion 304 becomes the angle αa+the angle αb. ha is a height measured from the foremost position Po of the edge tip portion 304 in which the edge tip surfaces 305a and the edge tip surface 305b cross each other on the center line L to the position Pa, and hb is a height measured from the position Po to the position Pb. Therefore, the height H of the edge tip portion 304 becomes the height ha+the height hb.

The blades 20 of the type having the angle a of about 40°: All the blades 20 that had edge tip portions A, B, C incised up to the corneal stromas (failed to incise and separate only the corneal epitheliums). In these blades 20, the edge tip surfaces 305b were left as extensions of the edge surfaces 303b, and the edge tip surfaces 305a were formed by polishing the edge surfaces 303a.

The blades 20 of the type having the angle a of about 60°: All the blades 20 that had edge tip portions A, B, C incised up to the corneal stromas (failed to incise and separate only the corneal epitheliums). In these blades 20, the edge tip surfaces 305b were left as extensions of the edge surfaces 303b, and the edge tip surfaces 305a were formed by polishing the edge surfaces 303a.

The blades 20 of the type having the angle a of about 80°: All the blades 20 that had edge tip portions A, B, C could excellently incise and separate the corneal epitheliums only. In these blades 20 as well, the edge tip surfaces 305a were formed by polishing the edge surfaces 303a, and the edge tip surfaces 305b were formed by polishing the edge surfaces 303b.

The blades 20 of the type having the angle a of about 100°: All the blades 20 that had edge tip portions A, B, C could excellently incise and separate the corneal epitheliums only. In these blades 20 as well, the edge tip surfaces 305a are formed by polishing the edge tip surfaces 303a, and the edge surfaces 305b were formed by polishing the edge surfaces 303b.

The blades 20 of the type having the angle a of about 120°: All the blades 20 that had edge tip portions A, B, C could excellently incise and separate the corneal epitheliums only. In these blades 20 as well, the edge tip surfaces 305a were formed by polishing the edge surfaces 303a, and the edge tip surfaces 305b were formed by polishing the edge surfaces 303b.

The blades 20 of the type having the angle α of about 140°: The blades 20 that had edge tip portions A, B only slipped on the corneal epitheliums, and could not incise the same. The blade 20 that has an edge tip portion C slipped on the cornea epitheliums in the hinge portion, and the corneal epitheliums remained but in the other portions could incise and separate the corneal epitheliums. In these blades 20 as well, the edge tip surfaces 305a were formed by polishing the edge surfaces 303a, and the edge tip surfaces 305b were formed by polishing the edge surfaces 303b.

The blades of the type having the angle α of about 160°: The blades 20 that had edge portions of A, B, C only slipped on the corneal epitheliums, and could not incise the same. In these blades 20 as well, the edge tip surfaces 305a were formed by polishing the edge surfaces 303a, and the edge tip surfaces 305b were formed by polishing the edge surfaces 303b.

These results of experiments show that, in order to incise the corneal epithelium, the angle α may be at least smaller than about 140°. It is further proven that the angle α is preferably not larger than about 120°. In order to separate the corneal epithelium only and not to incise up to the corneal stroma, it is proven that the angle α ay be not smaller then about 80°.

In view of the fact that, although the pig's eye does not have the Bowman's membrane, the human's eye has the Bowman's membrane considerably harder than the corneal stroma, it is considered that the corneal epithelium only can be separated without incising up to the corneal stroma even when the angle a is smaller than the above-mentioned level. According to the results of experiments in FIG. 5, since the blades 20 having the edge tip portions 304 of the type of the angle α° of about 60° has the edge tip surfaces 305b left as extensions of the edge surfaces 303b, the edge tip surfaces 305a only being formed by polishing the edge surfaces 303a, up to the corneal stromas may become easy to be incised. When the edge tip surfaces 305a are formed by polishing the edge surfaces 303a with the edge tip surfaces 305b formed by polishing the edge surfaces 303b, it is considered that separating of the corneal epitheliums only can be done without incising up to the corneal stromas even though the angle α is not smaller than 60°. Although it is preferable that the edge tip surface 305a and the edge tip surface 305b be substantially identical (symmetrical) with respect to the center line L, these angles may be slightly one-sided. It is also considered that separating of only the corneal epitheliums can be done without incising up to the corneal stroma when the height H is not smaller than about 3 μm. The height H is preferably not smaller than 5 μm. The reason resides in that, when the height H is too small, even the corneal epithelium is incised depending upon the smallness (sharpness) of the angle θ even though the angle α is large.

FIG. 8 is a diagram showing the results of further experiments for making sure of an allowable range of the angle α of the edge tip portion 304 having the first shape. The experiments were conducted by using pigs' eyes, dividing the angle α into two types of about 60° and about 70°, and prepare blades 20 having a plurality of kinds of edge tip portions 304. In these blades 20, the edge surfaces 305a were formed by polishing the edge surfaces 303a, and the edge tip surfaces 305b were formed by polishing the edge surfaces 303b.

The blades of the type having the angle α of about 60°: About a half of the all blades 20 could excellently incise and separate the corneal epitheliums only.

The blades of the type having the angle α of about 70°: Almost all of the blades 20 could excellently incise and separate the corneal epitheliums only.

Owing to these results of experiments, it was discovered that, when the edge tip surfaces 305a and 305b were formed substantially identically (symmetrically) with respect to the center line L, the corneal epithelium only could be separated without incising up to the corneal stroma even though the angle α was not smaller than about 60°. It was also discovered that, even when the angle α was not smaller than about 70°, more preferable effect was obtained.

From these results, it was also discovered that setting the angle αb of the lower edge tip surface 305b with respect to the center line L passing the distal end Po larger than the angle (holding angle (incising angle) of the blade 20 with respect to the horizontal plane) of the center line L with respect to the horizontal plane in the blade 20 at the time of incision and separation of the corneal epithelium is a preferable element for separating the corneal epithelium only without incising up to the corneal stroma.

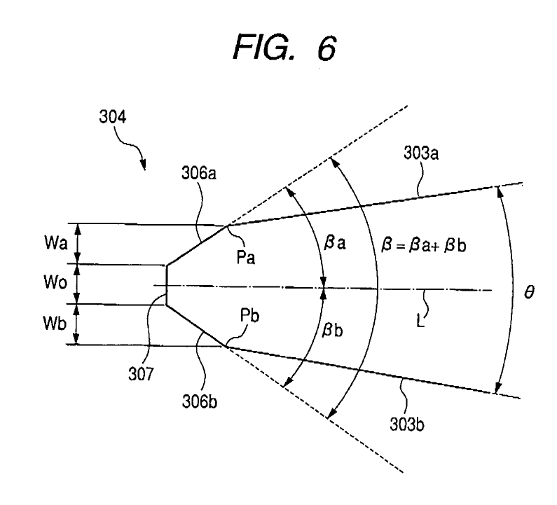

FIG. 6 is an enlarged view for describing a second shape of the edge tip portion 304 of the blade 20. The edge tip portion 304 having the second shape has a distal end flat surface 307 formed by polishing the distal end of the edge tip portion 304 having the first shape shown in FIG. 4. A height Wo of the distal end flat surface 307 is not smaller than about 1 μm and not larger than about 3 μm. An angle β formed between upper and lower edge tip surfaces 306a and 306b which form the edge tip portion 304 with the distal end flat surface 307 is not smaller than about 30° and not larger than about 80°, and preferably not smaller than about 50° and not larger than about 70°. A height Wa measured from an upper edge of the distal end flat surface 307 to a boundary position Pa between the edge tip surface 306b and the edge surface 303a, and a height Wb measured from a lower edge of the distal end surface 307 to a boundary position Pb between the edge tip surface 306b and the edge surface 303b are not smaller than about 2 μm.

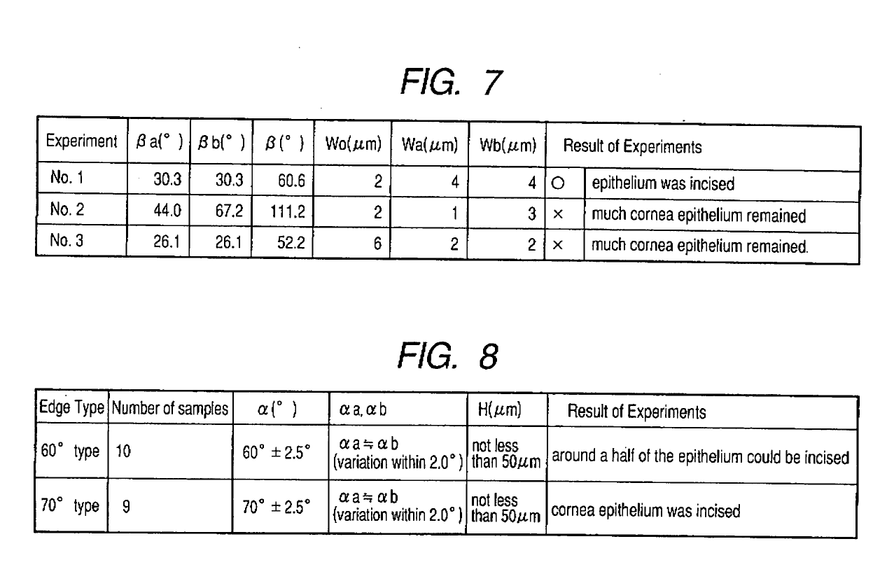

FIG. 7 is a diagram showing the results of experiments for making sure of an allowable range of the angle β of the edge tip portion 304 having the second shape. The experiments were conducted by using rabbit's eyes. The edge tip portion 304 in Experiments No. 1 had the height Wo of the distal end flat surface 307 of 2 μm, and the angle β of 60.6°, and could excellently incise and separate the corneal epitheliums only. The edge tip portion 304 in Experiments No. 2 had a height Wo of the distal end flat surface 307 of 2 μm, and the angle β of 111.2°, and much corneal epitheliums remained. The edge tip portion 304 in Experiments No. 3 had the height Wo of the distal end flat surface 307 of 6 μm, the angle β of 52.2°, and much corneal epitheliums remained.

Referring to FIG. 6 and FIG. 7, βa is an angle of the edge tip surface 306a with respect to the center line L, and βb is an angle of the edge tip surface 306b with respect to the center line L. Therefore, the angle β of the edge portion 304 becomes the angle βa+the angle βb.

In view of the above, it was proven that, as long as the distal end flat surfaces 307 having the height Wo of about 2 μm were formed, the blades 20 were possible separate the corneal epitheliums only without incising up to the corneal stromas even though the angle β is small (sharp) as compared with the angle α. Considering that the human eye has the Bowman's membrane harder than the corneal stroma, separating the corneal epithelium only without incising up to the corneal stroma is thought to be possible on the assumption that the height Wo is about 1 μm with the angle β about 30°. On the other hand, when the height Wo of the distal end flat surface 305 is set too large, the corneal epithelium becomes unable to be incised. When the angle β is set too large, the corneal epithelium becomes unable to be incised. Therefore, when the height Wo is not smaller than about 1 μm and not larger about 3 μm with the angle β not smaller than about 30° and not larger than about 80° and more preferably not smaller than about 50° and not larger than about 70°, it is considered possible to incise and separate the corneal epithelium only.

The operations using such an apparatus, for incising and separating the corneal epithelium will now be described. After the suction ring 31 is placed on the patient's eye, the pump 41 is operated, and the air in the space S between the suction ring 31 and the patient's eye is sucked to cause the air pressure to be reduced. The suction ring 31 is thereby suction-fixed to the patient's eye. In accordance with a signal from the foot switch 42, the control unit 40 rotates the motor 12 and the motor 11. As a result, the incising unit 2 moves linearly in the incising and separating direction while keeping the blade 20 laterally oscillating. The transaction speed of the blade 20 is set not lower than about 0.4 mm/sec to not higher than about 6 mm/sec and the number of oscillations about 5,000 rpm and not higher than about 25,000 rpm.

The corneal epithelium is incised with the edge tip portion 304 of the blade 20 moving linearly while laterally oscillating. The edge tip portion 304 then reaches the Bowman's membrane under the corneal epithelium but not incise up to the Bowman's membrane harder than the corneal epithelium, the blade 20 moving straight so as to slip on the Bowman's membrane. As a result, the cornea epithelium is separated from the Bowman's membrane (or basement membrane of the epithelium), and a flap is formed.

In the above-mentioned embodiment, the blade 20 translated linearly is used but the blade 20 translated rotationally may be used. Only lateral oscillation of the blade may be made by a power source, such as a motor, and linear movement or rotational movement thereof may be made manually.

What is claimed is:

1. A blade for used in corneal surgery for incising and separating a corneal epithelium into a flap shape, the blade comprising:
an edge tip portion including a distal end and two upper and lower edge tip surfaces,
wherein an angle formed between the two edge tip surfaces is not smaller than 60° and smaller than 140°, and
wherein a height of the edge tip portion is not smaller than 3 μm.

2. The blade according to claim 1, wherein the angle formed between the two edge tip surfaces is not smaller than 70° and not larger than 120°.

3. The blade according to claim 1, wherein an angle of the lower edge tip surface with respect to a center line passing the distal end is larger than an angle of the center line with respect to a horizontal plane in the blade at the time of incision and separation of the corneal epithelium.

4. A corneal surgical apparatus comprising:
holding means for oscillatingly holding the blade according to claim 1; and
oscillating means for oscillating the blade, wherein the corneal epithelium is incised and separated with the oscillating blade.

5. A corneal surgical apparatus comprising: an incising unit provided with a blade holder for holding the blade according to claim 1 oscillatingly in a predetermined direction; and a vibrating mechanism that has a driving power source, converts power from the power source into kinetic energy in a predetermined direction, transmits the kinetic energy to the blade, and oscillates the blade, wherein the corneal epithelium is incised and separated with the oscillating blade.

6. A blade for used in a corneal surgery, for incising and separating a corneal epithelium into a flap shape, the blade comprising:
an edge tip portion including a distal end flat surface, and two upper and lower edge tip surfaces,
wherein an angle formed between the two edge tip surfaces is not smaller 30° and smaller than 80°, and
wherein a height of the distal end flat surface is not smaller than 1 μm and not larger than 3 μm.

7. The blade according to claim 6, wherein the angle formed between the two edge tip surfaces is not smaller than 50° and not larger than 70°.

8. A corneal surgical apparatus comprising: holding means for oscillatingly holding the blade according to claim 6; and oscillating means for oscillating the blade, wherein the corneal epithelium is incised and separated with the oscillating blade.

9. A corneal surgical apparatus comprising: an incising unit provided with a blade holder for holding the blade according to claim 6 oscillatingly in a predetermined direction; and a vibrating mechanism that has a driving power source, converts power from the power source into kinetic energy in a predetermined direction, transmits the kinetic energy to the blade, and oscillates the blade, wherein the corneal epithelium is incised and separated with the oscillating blade.
